(19) United States
(12) Patent Application Publication
(10) Pub. No.: US 2014/0155726 A1
(43) Pub. Date: Jun. 5, 2014

(54) Dynamic tonometry device and method for controlling coaxiality of probe with eyeball

(71) Applicant: HUAINAN NORMAL UNIVERSITY, Huainan, Anhui (CN)

(72) Inventors: Jianguo Ma, Huainan, Anhui (CN); Jin Zhang, Huainan, Anhui (CN); Yijie Wang, Huainan, Anhui (CN); Ming Liu, Huainan, Anhui (CN)

(73) Assignee: HUAINAN NORMAL UNIVERSITY, Huainan, Anhui (CN)

(21) Appl. No.: 14/232,262

(22) PCT Filed: Jan. 7, 2013
(86) PCT No.: PCT/CN2013/070153

§ 371 Date: Jan. 11, 2014

(30) Foreign Application Priority Data
Aug. 6, 2012 (CN) 201210284491.8

(51) Int. Cl. A61B 3/16; A61B 3/00; A61B 3/14

(52) U.S. Cl. CPC A61B 3/16; A61B 3/14; A61B 3/0008; A61B 3/0025
USPC 600/399

(57) ABSTRACT

A dynamic tonometry device includes a probe, a housing, a sleeve, a light source, an image sensor, a pressure sensor, a microprocessor and a display storage. The probe is a truncated cone with small at left and large at right. The sleeve is fitted on the probe. An end face of a small end of the probe is situated to a left of a left end face of the sleeve. A right end of the sleeve is connected to a left end of the housing; on a large end of the probe is installed the pressure sensor; inside the housing are installed the light source and the image sensor. Light emitted by the light source is collimated into a light beam which is incident to the probe and totally reflected before entering the image sensor. With the microprocessor are connected the pressure sensor, the image sensor and the display storage.

### CROSS REFERENCE OF RELATED APPLICATION

This is a U.S. National Stage under 35 U.S.C 371 of the International Application PCT/CN2013/070153, filed Jan. 7, 2013, which claims priority under 35 U.S.C. 119(a-d) to CN 201210284491.8, filed Aug. 6, 2012.

### BACKGROUND OF THE PRESENT INVENTION

1. Field of Invention

The present invention relates to a dynamic tonometry device, and more particularly to a contact dynamic tonometry device and a method for controlling a coaxiality of a probe thereof with an eyeball by the contact dynamic tonometry device.

2. Description of Related Arts

Intraocular pressure is often closely related with various eye diseases. Currently, glaucoma is the world's second largest irreversible blinding eye disease. According to statistics, there are around more than 67 million patients with primary glaucoma worldwide. In China, there are currently at least 5 million glaucoma patients, and among them 790 thousand persons lose the sight of both eyes. The prevalence rate of this eye disease increases with age. Glaucoma is characterized by pathological intraocular pressure increase, irreversible optic atrophy and visual field defect, making a serious impact on the quality of life of patients. In China, glaucoma, with incidence rate of 0.21%-1.64% and blindness rate of 10%-20%, is one of major diseases which endanger the health of the middle-aged and elderly (55-70 years old). In order to prevent glaucoma, the most common and the most effective method is to measure the intraocular pressure of patients and to control the intraocular pressure increase by means of medication.

Intraocular pressure refers to a size of pressure per unit volume when the eyeball contents (aqueous humor, lens, vitreous body, blood) act on the wall of the eyeball. Long-term intraocular pressure increase can lead to optic nerve ischemia and tolerance lowering at the same level of intraocular pressure, causing neurodegeneration. Moreover, electrical signals converted by the retina can not smoothly pass and stimulate the visual center of occipital lobe of the brain, eventually resulting in the corresponding irreversible visual field defects. For the traditional intraocular pressure measurement by tonometer, there are two methods, namely, implanting type and non-implanting type. It is difficult for the implanting type to have clinical operability, although it can measure directly intraocular pressure. For this reason, the non-implanting indirect measurement method has to be relied on in clinical application. Tonometers in a usual sense can be defined as those for the non-implanting indirect measurement. For today's dominant non-implanting indirect measurement, there are mainly two kinds of tonometers, one being indentation tonometer, the other applanation tonometer. For indentation tonometer, airflow is usually ejected through the end of the probe and reaches the eyeball, so that intraocular pressure is obtained at the moment when the eyeball is indented. Because in a real sense there exists no instruments which are in direct contact with the eyeball, this method can avoid not only the cross-infection of some diseases, but also the anesthesia of the eye cornea. However, because of its high cost, lack of good accuracy, higher demand for the operator's operating skills, eventual unnecessary injuries to the cornea, and requirement of frequent maintenance, this method can not be widely used for clinical application. For applanation tonometer, a probe is pressed against the outer portion (such as cornea) of eyeball in so far as a certain area, and corresponding pressure is acquired, thereby obtaining intraocular pressure. The representative applanation tonometer is the Goldmann applanation tonometer, which is considered the “gold standard”.

Because the existing intraocular pressure testing instruments are generally not able to judge very well whether an axis of a measuring contact of a testor is coincident with a longitudinal axis of an eyeball, the results of intraocular pressure testing present larger error. Moreover, an operator is demanded a higher proficiency in the operation, which is required to be completed by a professional ophthalmologist for patients. And because the alignment operation of intraocular pressure testing instruments is difficult and time-consuming, the measurement is not easily carried out for patients with low degree of endurance, and the measurement error is large.

### SUMMARY OF THE PRESENT INVENTION

An object of the present invention is to provide a dynamic tonometry device which is simple to manipulate with high accuracy of measurement, is able to quickly complete the measurements, and is able to achieve accurate measurements even for patients with low degree of endurance.

A dynamic tonometry device of the present invention comprises: a probe, a housing, a sleeve, a light source, an image sensor, a pressure sensor, a microprocessor, a display storage and a power source, the probe taking the form of truncated cone with small at the left and large at the right and being made of transparent optical materials; an inner hole of the sleeve being in the same form as the probe; the sleeve being slidably fitted on the probe; an end face of a small end of the probe being situated to the left of a left end face of the sleeve; a right end of the sleeve being fixedly connected with a left end of the housing; on a large end of the probe being installed the pressure sensor; a sensing end of the pressure sensor being pressed onto a left end face of the housing; inside the housing being installed the light source, the image sensor and a convex lens; light emitted by the light source being collimated through the convex lens into a parallel light beam, and then the light beam being vertically incident to the large end of the probe; after undergoing total reflection in the probe, the light beam entering into the image sensor; inside the housing being installed the microprocessor, the display storage and the power source; with the power source being connected the microprocessor, the display storage, the pressure sensor, the image sensor and the light source; and, with the microprocessor being connected the pressure sensor, the image sensor and the display storage.

In the dynamic tonometry device of the present invention, a central axis of the convex lens coincides with an axis of the probe.

In the dynamic tonometry device of the present invention, the light source and the image sensor are respectively located on both sides of the axis of the probe and are disposed symmetrically with respect to the axis of the probe.

In the dynamic tonometry device of the present invention, on an inner wall of the left end of the housing is fixedly mounted an annular metallic press-ring; the pressure sensor is an annular electric pressure sensor; at a joint portion between a right end face and a circumferential face of the probe is provided an annular groove, inside which the pressure sensor is fixedly mounted; and the sensing end of the pressure sensor is in contact with the annular metallic press-ring.

In the dynamic tonometry device of the present invention, the light source is a light emitting diode.

In the dynamic tonometry device of the present invention, the probe is made of glass or resin.

The dynamic tonometry device of the present invention further comprises a speaker which is fixedly mounted inside the housing and is connected with the microprocessor.

In the dynamic tonometry device of the present invention, the light source is also provided on its left side with a wave filter.

The dynamic tonometry device of the present invention further comprises a green optical filter which is fixedly disposed in the central axis of the probe and is located at the left side of the light source.

The dynamic tonometry device of the present invention differs from the prior art as follows. According to the present invention, the light is emitted by the light source; when the probe is close to the eyeball and the central point of the left end face of the probe is not in contact with the apex point of the quasi-dome-shaped cornea of the eyeball, if the parallel light is incident from the probe, optically denser medium, to the air, optically thinner medium, then total reflection occurs; after the first total reflection occurs at the surface of a side of the probe, the parallel light is directed towards the left end face of the probe where the second total reflection occurs; then the light beam reaches the surface of another side of the probe, total reflection occurs again; finally, the light emitted by the light source is reflected to the image sensor which detects it as bright spot; when the central point of the left end face of the probe begins to be in contact with the apex point of the quasi-dome-shaped cornea of the eyeball, at this time, the location of contact with the probe is the eyeball, optical media is changed from the air to the eyeball, the refractive index varies, accordingly, the conditions of occurrence of total reflection are not satisfied; the light at the central point of the left end face of the probe is incident to the eyeball, meanwhile, the light at the position of contact with the eyeball of the probe does not enter into the image sensor, the image sensor detects dark half-loop or loop line appearing on the bright spot; when the probe continues to be depressed, applanation surface gradually increases, the image sensor detects half-loop or loop applanation image with loop width gradually increasing, meanwhile, loop width of the half-loop or loop applanation image is uniformized in order to ensure the coincidence of the axis of the probe with the longitudinal axis of the eyeball. If it is calculated by the microprocessor that loop width is nonuniform, then on the display memory is displayed a prompt that the axes do not coincide, at this time the position of the probe can be quickly adjusted for the purpose of the coincidence of the axes; during the depression of the probe, the image sensor and the pressure sensor can respectively measure the active applanation surfaces and applanation forces, which pass through the microprocessor and then are displayed and stored by the display storage. According to the present invention, it can be determined that the axis of the probe coincides with the longitudinal axis of the eyeball by simply observing the content displayed on the display storage. This device is simple to manipulate with high accuracy of measurement, is able to quickly complete the measurements, and can achieve accurate measurements even for patients with low degree of endurance.

Another object of the present invention is to provide a method for controlling a coaxiality of a probe axis of the dynamic tonometry device mentioned above with a longitudinal axis of an eyeball, wherein the method comprises steps of:

(a) turning on the power source to supply the tonometry device with electricity;

(b) aligning perpendicularly the probe with a top of an eye cornea and aligning a central point of a left end face of the probe with an apex point of a dome-shaped cornea;

(c) depressing slowly the probe, along with a gradual increase of applanation force, on the display storage being displayed a half-loop or loop applanation image; and

(d) uniformizing a loop width of the half-loop or loop applanation image.

The method of the present invention quickly obtains the coaxiality of the axis of the probe with the longitudinal axis of the eyeball, thereby achieving accurate and quick measurement of the applanation surface and the applanation force.

### BRIEF DESCRIPTION OF THE DRAWINGS

The present invention will be described further herein below with reference to the accompanying drawings, in which:

FIG. 1 is a front view of a dynamic tonometry device according to a first embodiment of the present invention;

FIG. 2 is a partial enlarged view of a probe of FIG. 1.

FIG. 3a is a real applanation image when a central point of a left end face of the probe is in contact with an apex point of a quasi-dome-shaped cornea of an eyeball according to the first embodiment of the present invention;

FIG. 3b is a half-loop applanation image displayed on a display storage when the probe is pressed against the eyeball in FIG. 3a;

FIG. 4a is a real applanation image when the left end face of the probe is pressed further against the quasi-dome-shaped cornea of the eyeball (a diameter of the real applanation image being 2 mm) according to the first embodiment of the present invention;

FIG. 4b is a half-loop applanation image displayed on the display storage when the probe is pressed against the eyeball in FIG. 4a;

FIG. 5a is a real applanation image when the left end face of the probe is pressed further against the quasi-dome-shaped cornea of the eyeball (the diameter of the real applanation image being 4 mm) according to the first embodiment of the present invention;

FIG. 5b is a half-loop applanation image displayed on the display storage when the probe is pressed against the eyeball in FIG. 5a;

FIG. 6a is a real applanation image when the left end face of the probe is depressed further against the quasi-dome-shaped cornea of the eyeball (the diameter of the real applanation image being 6 mm) according to the first embodiment of the present invention;

FIG. 6b is a half-loop applanation image displayed on the display storage when the probe is depressed against the eyeball in FIG. 6a;

FIG. 7 is a circuit diagram of the dynamic tonometry device according to the first embodiment of the present invention;

FIG. 8 is a front view of the dynamic tonometry device according to a second embodiment of the present invention.

### DETAILED DESCRIPTION OF THE PREFERRED EMBODIMENT

### First Embodiment

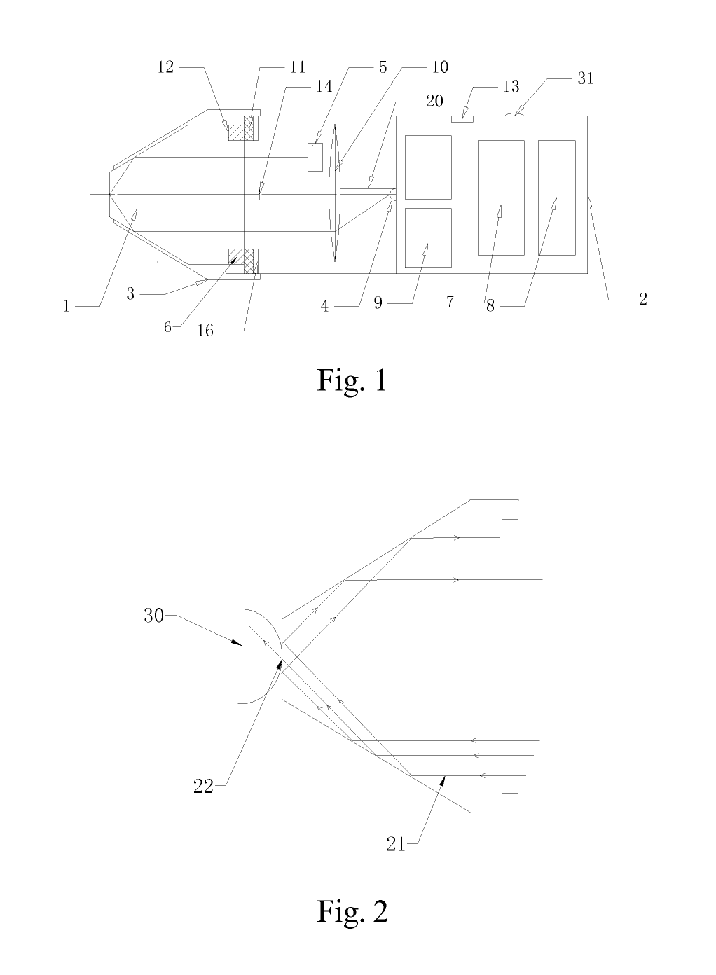

As shown in FIG. 1, a dynamic tonometry device of the present invention comprises: a probe 1, a housing 2, a sleeve 3, a light source 4, an image sensor 5, a pressure sensor 6, a microprocessor 7, a display storage 8, a green optical filter 14, a speaker 13, and a power source 9.

The probe 1 takes a form of a truncated cone with small at left and large at right, and is made of transparent optical materials. Conditions under which light is totally transmitted at a side and a bottom of the probe 1 are relevant with an incident angle of the light and with the materials of the probe. When the incident angle is greater than or equal to a critical angle, if the light is directed from inside the probe to the side or the bottom of the probe, total reflection occurs. Therefore, the conditions under which the total reflection occurs inside the probe 1 are the incident angle and the critical angle, which are determined by the material selected for the probe. The critical angle varies with the material. For example, in the first embodiment, glass K9 is used for the probe 1. The angle between an axis of the truncated cone and a generatrix of the truncated cone of the probe 1 is set to 29 degrees in order to satisfy requirements of the total reflection at the side and the bottom of the probe 1. If other materials are selected for the probe 1, then according to different refractive indexes of the materials, the angle between the axis of the truncated cone and the generatrix of the truncated cone of the probe 1 changes accordingly. A left end surface of the probe 1 has a diameter of 6 mm. In the first embodiment, an outer peripheral portion of a right end of the probe 1 is partly machined away so as to form a left half part of the form of the truncated cone and the right half part of a form of a cylinder. At a joint portion between a right end face and a circumferential face of the probe 1 is provided an annular groove 12, inside which the pressure sensor 6 is fixedly mounted. The pressure sensor 6 is an annular electric pressure sensor. However, other annular pressure sensors can also be used.

An inner hole of the sleeve 3 is in the same form as the probe 1, on which the sleeve 3 is fitted. The probe 1 is able to slide axially inside the sleeve 3. At the time of measurement, there exists almost no friction or a considerably low friction to a negligible level between the sleeve 3 and the probe 1. An end face of a small end of the probe 1 is situated to a left of a left end face of the sleeve 3, and a right end of the sleeve 3 is screw-fixedly connected to a left end of the cylindrical housing 2. At a left end of an inner cavity of the housing 2 is disposed an annular boss 16, at a left end face of which is fixedly mounted an annular metallic press-ring 11. The annular metallic press-ring 11 is disposed oppositely to the groove 12 provided on the right end face of the probe 1 and is in contact with a sensing end of the pressure sensor 6.

In order to prevent virus infection, that is to say, for example, the prions (i.e., proteinaceous infectious particles) which have been found in lachrymals are infectious and can pass through lachrymal contact from the eyes of a person to another person, and practice has proved that an infected object is not easily sterilized, for this reason, the probe 1 is mounted inside the sleeve 3, and after each measurement is complete, the sleeve 3 is unscrewed from the housing 2, then the probe 1 can be easily replaced. The probe 1 is made of optical glass. For the purpose of cost reduction, resin of low cost can be selected as the material for manufacturing the probe 1.

The light source 4, a convex lens 10, the image sensor 5, the pressure sensor 6, the microprocessor 7, the display storage 8, the green optical filter 14, the speaker 13 and the power source 9 are respectively fixedly mounted inside the housing 2. In the first embodiment, the light source 4 is situated at a focal point of a right side of the convex lens 10. The power source 9 is located at an axis of the probe 1. The green optical filter 14 is located at the axis of the right side of the probe 1 and at the left side of the convex lens 10. The image sensor 5 is situated above the axis of the probe 1. In the first embodiment, within the housing 2 is also fixedly mounted a baffle 20, which is located above the light source 4 so that the light emitted from the light source 4 enters into only the lower half of the convex lens 10, thereby obtaining a half-loop applanation image. Of course, it is not required to dispose the baffle 20, thereby obtaining a loop applanation image. The light source 4 may be a light emitting diode, an incandescent lamp or a fluorescent lamp, which emits visible light, and may be a point light source, a linear light source or an annular light source. Thanks to its excellent stability, efficiency and longevity, light emitting diode is used as the light source 4 in the first embodiment. On a left side of the light source 4 is also disposed a wave filter (not shown in the drawings), which is able to match a wavelength of the light incident to the probe 1 to a receiving wavelength range necessary for the image sensor 5. The image sensor 5 may be a monochrome or color CCD or CMOS device and uses a one-dimensional linear device, which includes an analysis circuit for collecting geometric parameters passing through the loop applanation image, such as radius or loop width. The light emitted by the light source 4 is collimated through the convex lens 10 into a parallel light beam, and then the light beam is vertically incident to the large end of the probe 1. The light beam performs three total reflections inside the probe 1 and then enters into the image sensor 5. With reference to FIG. 7, the microprocessor 7, the display storage 8, the pressure sensor 6, the image sensor 5 and the light source 4 are connected with the power source 9, and moreover, the pressure sensor 6, the image sensor 5 and the display storage 8 are connected with the microprocessor 7. The microprocessor 7 assumes a responsibility for monitoring and calculating all data provided by the image sensor 5 and the pressure sensor 6. The display storage 8 is connected to the microprocessor 7, in such a manner that intraocular pressure values obtained by treatment and calculation are displayed and stored. A visual panel of the display storage 8 is located on the housing 2, in order to facilitate an observation by a measurement person. Based on actual needs, the display memory 8 can be set so as to display the images or display simultaneously both the images and the intraocular pressure data.

The dynamic tonometry device of the present invention works on the following principle.

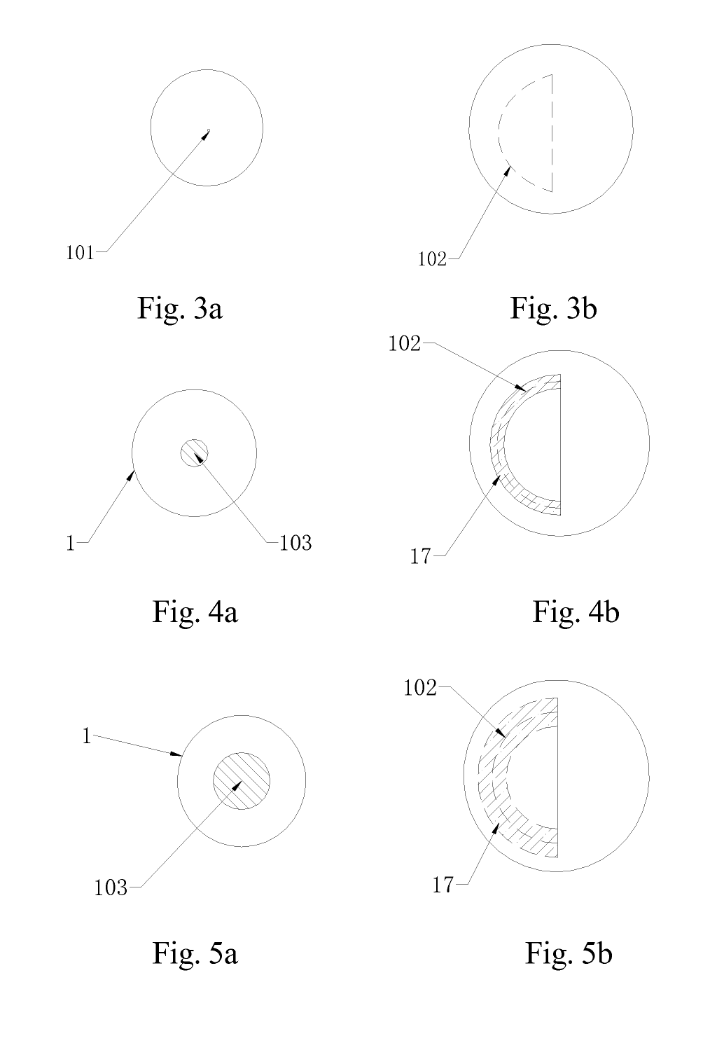

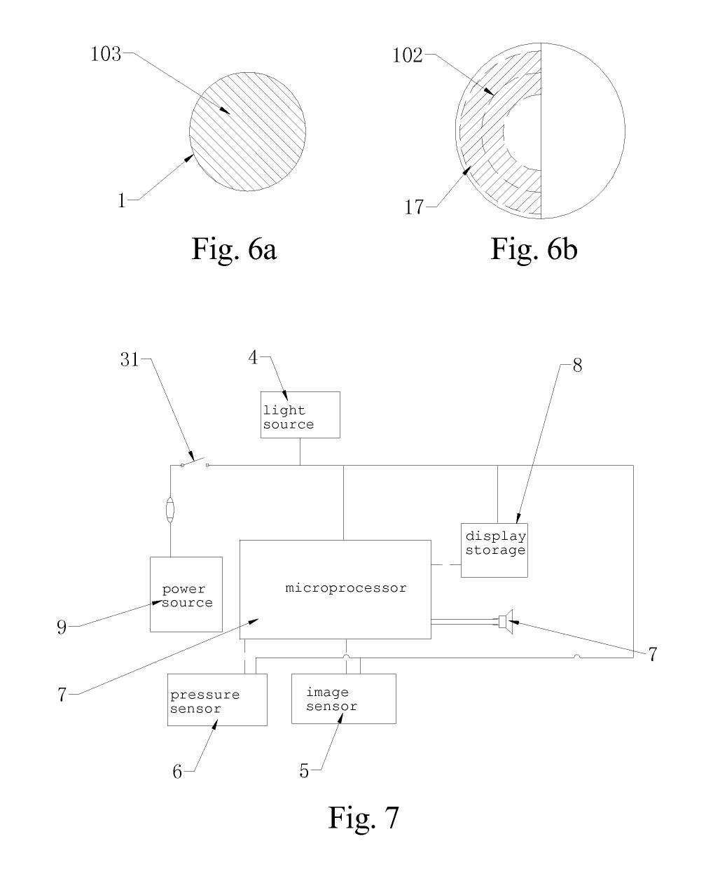

As shown in FIG. 2, a portion of the light emitted by the light source 4 (i.e. light, of which the reverse extension line passes through the focal point of the convex lens) is collimated through the convex lens 10 to form a parallel beam 21, which is currently parallel to the axis of the probe 2. The parallel light beam 21 is incident from the right end of the probe 1, performs a total reflection at the lower side surface of the probe 1, and then is directed to the left end face of the probe 1, where a second total reflection occurs. Then, the light beam reaches the upper side surface of the probe 1, and a total reflection occurs again. The light emitted by the light source 4 is reflected on the image sensor 5, and its image is displayed as bright spot. The light which is emitted by the light source 4 and is not collimated by the convex lens 10 into a parallel beam, either is attenuated and disappears after repeated reflections inside the probe 1, or does not meet the conditions for total reflection and is emergent from the probe 1. Only a very small amount of light becomes the disturbing light and enters into the image sensor 5. When the central point 22 of the left end face of the probe 1 comes into contact with the eyeball 30, as shown in FIG. 3a, an applanation image of the contact portion is a contact point 101. An applanation image coming from the right side of the probe 1 and detected by the image sensor 5 detects, as shown in FIG. 3b, is displayed as a dark half-loop line 102. However, except for this, the rest in the entire field of view is bright. This is because the light of the rest except the contact point 101 can be totally reflected and is displayed as bright spot, and only the light of the contact point 101 can enter into the eyeball. As shown in FIG. 2, since the light of the middle of a parallel light beam 21 enters into the eyeball and the light of both sides of the parallel light beam 21 enters through total reflection into the image sensor 5, the image detected by the image sensor 5 is a dark half-loop line 102. As the pressure increases, as shown in FIG. 4a, the contact portion between the probe 1 and the cornea of the eyeball changes from the contact point 101 to the contact plan 103, and moreover, the surface (applanation surface) of this contact plan can gradually increase. The light originally being totally reflected on this contact plan concerned enters now almost entirely into the eyeball, and produces an applanation image which is no longer just a dark half-loop line 102, but is as shown in FIG. 4b a half-loop applanation image 17 of a certain width. This half-loop applanation image 17 is captured by the image sensor 5, and transmitted to the microprocessor 7. As applanation force increases, contact plan between the probe 1 and the cornea will gradually increase, therefore loop width of the half-loop applanation image 17 resulting therefrom becomes wider and wider along with the increasing applanation force. As shown in FIGS. 5a, 5b, the contact plan 103 increases, and the half-loop applanation image 17 presents such a characteristic that it gradually spreads to both sides by taking the original dark half-loop line 102 as the central axis. When the contact plan 103 increases to the situation shown in FIG. 6a, the contact plan between the probe 1 and the cornea reaches the maximum, namely, applanation surface reaches the maximum. With the increase of applanation force, applanation surface will no longer increase accordingly. As shown in FIG. 6b, at this time the half-loop applanation image 17 reaches the maximum, i.e. loop width also reaches the corresponding maximum value. In the course of measurement, applanation surface is obtained by performing a continuous dynamic detection of the width of the half-loop applanation image 17, and by utilizing the linear relationship between loop wide and applanation surface (the contact plan), as with in this embodiment the relationship between loop width of the half-loop applanation image 17 and radius of the contact plan 103. Meanwhile, the corresponding applanation force obtained by the pressure sensor 6 is recorded, then the intraocular pressure value (i.e. value obtained by dividing applanation force by applanation surface) is calculated by the microprocessor 7, and is displayed and storaged by the display storage 8.

However, in the course of measurement, the axis of the probe 1 deviates from the longitudinal axis of the eyeball, and then a great impact will be given to the result of intraocular pressure, thereby leading to unnecessary error. For this reason, at the time of measurement, the result measured only when there exists the coaxiality of the axis of the probe 1 with the longitudinal axis of the eyeball is closest to the true value of intraocular pressure, and only in this case, the ulterior measurement process can be started. Therefore, it is necessary first of all to determine whether there exists the coaxiality. The method thereof comprises steps of:

(a) turning on the power source 9 to supply the tonometry device with electricity;

(b) aligning perpendicularly the probe 1 with a top of an eye cornea and aligning the central point 22 of the left end face of the probe 1 with an apex point of the dome-shaped cornea;

(c) depressing slowly the probe 1, along with a gradual increase of applanation force, on the display storage 8 being displayed a half-loop or loop applanation image 17; and

(d) uniformizing a loop width of the half-loop or loop applanation image 17.

When the baffle 20 is disposed, the light emitted from the light source 4 enters into only the lower half of the convex lens, thereby forming a half-loop applanation image.

Then it is possible to make a judgment through the built-in programs of the microprocessor 7 and to issue a prompt through the speaker 13, or to carry out an observation through the display storage 8. If the coaxial conditions are satisfied, it is possible to start the collection and recording of data. If the requirements are not met, then re-measurement is needed. Therefore, it is possible to avoid unnecessary error and effectively solve the problem that multiple measurements cannot have an excellent consistency due to the coaxial deviation generally present in the conventional portable tonometers, thereby obtaining accurate results.

The dynamic tonometry device of the present invention is used and executed according to the following steps:

First step: press the power switch 31, to supply the respective parts with corresponding voltage; let a tested person to watch a beam of green light which is converted by the green optical filter 14; align the probe 1 with a top of a dome-shaped cornea on the pupil of the test person; carry out a fine adjustment of vertical direction of the probe 1 according to the image in the display storage 8 so that the probe 1 and the eyeball are in the same line to facilitate accurate measurement of intraocular pressure;

Second step: the operator brings the probe 1 slowly and vertically into contact with the cornea, at this time the image sensor 5 collects the data corresponding to the requirements and transmits it to the microprocessor 7, meanwhile the microprocessor 7 issues a command so that the corresponding pressure data is collected; in the course of depressing downward, this device will continue to collect the data which meets the requirements; in this process, intraocular pressure results corresponding to each set of data are displayed on the display storage 8 and temporarily stored by its storage system.

Third step: the microprocessor 7 calculates the corresponding intraocular pressure values, and moreover records and displays in real time applanation surface, applanation force, intraocular pressure value in the entire process of implementation of measurement.

In the case of medical clinical use, six sets of data required may be collected, and voice speaker 13 prompts that the data collection is completed. The six results which meet the requirements after completion of collection are averaged, and finally storaged and displayed.

### Second Embodiment

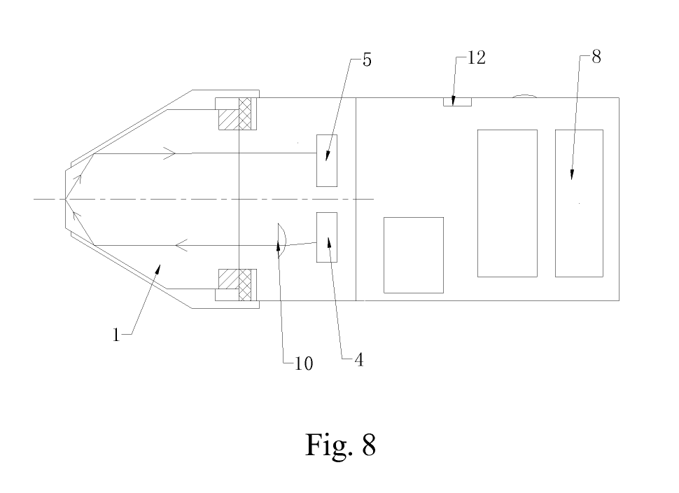

As shown in FIG. 8, the second embodiment is different from the first embodiment only in that: there is no baffle; the light source 4 is positioned below the image sensor 5; and the convex lens 10 is disposed on the left side of the light source 4. The light emitted by the light source 4 is collimated through the convex lens 10 into a parallel light beam, carries out total reflection inside the probe 1, and then is directly incident into the image sensor 5. In the second embodiment, a prompt is given by the speaker 12, or an observation is carried out through the display storage 8 to determine whether there exists the coaxiality of the axis of the probe 1 with the longitudinal axis of the eyeball.

The embodiments described above are described only as the preferred embodiments of the present invention and are not intended to limit the scope of the present invention. Under the premise of not departing from the spirit of the design of the present invention, a variety of changes and modifications made by ordinary skill in the art with respect to the technical schemes of the present invention shall fall within the scope of protection as disclosed in the accompanying claims of the present invention.

### INDUSTRIAL APPLICABILITY

According to the present invention, at the time of use, it is possible to ensure the coaxiality of the axis of the probe with the longitudinal axis of the eyeball by controlling the image sensor to detect the half-loop or loop applanation image and by uniformizing loop width of the half-loop or loop applanation image. If the microprocessor calculates the non-uniform loop width, then on the display storage is displayed a prompt that the axes do not coincide. At this time the position of the probe can be quickly adjusted for the purpose of the coincidence of the axes. During the depression of the probe, the image sensor and the pressure sensor can respectively measure the active applanation surfaces and applanation forces, which pass through the microprocessor and then are displayed and stored by the display storage. With the device of the present invention, at the time of measurement, it can be determined that the axis of the probe coincides with the longitudinal axis of the eyeball by simply observing the prompts displayed on the display storage. This device is simple to manipulate with high accuracy of measurement, is able to quickly complete the measurements, and can achieve accurate measurements even for patients with low degree of endurance. Therefore, this device has a great market prospect and a strong industrial applicability.

1-10. (canceled)

11. A dynamic tonometry device, comprising a probe, a housing, a sleeve, a light source, an image sensor, a pressure sensor, a microprocessor, a display storage and a power source, the probe taking a form of a truncated cone with small at left and large at right and being made of transparent optical materials; an inner hole of the sleeve being in the same form as the probe; the sleeve being slidably fitted on the probe; an end face of a small end of the probe being situated to a left of a left end face of the sleeve; a right end of the sleeve being fixedly connected with a left end of the housing; on a large end of the probe being installed the pressure sensor; a sensing end of the pressure sensor being pressed against a left end face of the housing; inside the housing being installed the light source, the image sensor and a convex lens; light emitted by the light source being collimated through the convex lens into a parallel light beam, and then the light beam being vertically incident to the large end of the probe; after performing total reflection inside the probe, the light beam entering into the image sensor; inside the housing being installed the microprocessor, the display storage and the power source; with the power source being connected the microprocessor, the display storage, the pressure sensor, the image sensor and the light source; with the microprocessor being connected the pressure sensor, the image sensor and the display storage.

12. The dynamic tonometry device according to claim 11, wherein a central axis of the convex lens coincides with an axis of the probe.

13. The dynamic tonometry device according to claim 11, wherein the light source and the image sensor are respectively located on both sides of an axis of the probe.

14. The dynamic tonometry device according to claim 11, wherein on an inner wall of the left end of the housing is fixedly mounted an annular metallic press-ring; the pressure sensor is an annular electric pressure sensor; at a joint portion between a right end face and a circumferential face of the probe is provided an annular groove, inside which the pressure sensor is fixedly mounted; and the sensing end of the pressure sensor is in contact with the annular metallic press-ring.

15. The dynamic tonometry device according to claim 12, wherein on an inner wall of the left end of the housing is fixedly mounted an annular metallic press-ring; the pressure sensor is an annular electric pressure sensor; at a joint portion between a right end face and a circumferential face of the probe is provided an annular groove, inside which the pressure sensor is fixedly mounted; and the sensing end of the pressure sensor is in contact with the annular metallic press-ring.

16. The dynamic tonometry device according to claim 13, on wherein an inner wall of the left end of the housing is fixedly mounted an annular metallic press-ring; the pressure sensor is an annular electric pressure sensor; at a joint portion between a right end face and a circumferential face of the probe is provided an annular groove, inside which the pressure sensor is fixedly mounted; and the sensing end of the pressure sensor is in contact with the annular metallic press-ring.

17. The dynamic tonometry device according to claim 14, wherein the light source is a light emitting diode.

18. The dynamic tonometry device according to claim 15, wherein the light source is a light emitting diode.

19. The dynamic tonometry device according to claim 16, wherein the light source is a light emitting diode.

20. The dynamic tonometry device according to claim 17, wherein the probe is made of glass or resin.

21. The dynamic tonometry device according to claim 18, wherein the probe is made of glass or resin.

22. The dynamic tonometry device according to claim 19, wherein the probe is made of glass or resin.

23. The dynamic tonometry device according to claim 20, further comprising a speaker which is fixedly mounted inside the housing and is connected with the microprocessor.

24. The dynamic tonometry device according to claim 21, further comprising a speaker which is fixedly mounted inside the housing and is connected with the microprocessor.

25. The dynamic tonometry device according to claim 23, wherein the light source is also provided on a left side with a wave filter.

26. The dynamic tonometry device according to claim 24, wherein the light source is also provided on its left side with a wave filter.

27. The dynamic tonometry device according to claim 11, further comprising a green optical filter which is fixedly disposed in a central axis of the probe and is located at a left side of the light source.

28. A method for controlling a coaxiality of a probe axis of the dynamic tonometry device, according to claim 11, with a longitudinal axis of an eyeball, comprising steps of:
(a) turning on the power source to supply the dynamic tonometry device with electricity;
(b) aligning perpendicularly the probe with a top of an eye cornea and aligning a central point of a left end face of the probe with an apex point of a dome-shaped cornea;
(c) depressing slowly the probe, along with a gradual increase of applanation force, on the display storage being displayed a half-loop or loop applanation image; and
(d) uniformizing a loop width of the half-loop or loop applanation image.

29. A method for controlling a coaxiality of a probe axis of the dynamic tonometry device, according to claim 25, with a longitudinal axis of an eyeball, comprising steps of:
(a) turning on the power source to supply the dynamic tonometry device with electricity;
(b) aligning perpendicularly the probe with a top of an eye cornea and aligning a central point of a left end face of the probe with an apex point of a dome-shaped cornea;
(c) depressing slowly the probe, along with a gradual increase of applanation force, on the display storage being displayed a half-loop or loop applanation image; and
(d) uniformizing a loop width of the half-loop or loop applanation image.

30. A method for controlling a coaxiality of a probe axis of the dynamic tonometry device, according to claim 26, with a longitudinal axis of an eyeball, comprising steps of:
(a) turning on the power source to supply the dynamic tonometry device with electricity;
(b) aligning perpendicularly the probe with a top of an eye cornea and aligning a central point of a left end face of the probe with an apex point of a dome-shaped cornea;
(c) depressing slowly the probe, along with a gradual increase of applanation force, on the display storage being displayed a half-loop or loop applanation image; and
(d) uniformizing a loop width of the half-loop or loop applanation image.

31. A method for controlling a coaxiality of a probe axis of the dynamic tonometry device, according to claim 27, with a longitudinal axis of an eyeball, comprising steps of:
(a) turning on the power source to supply the dynamic tonometry device with electricity;
(b) aligning perpendicularly the probe with a top of an eye cornea and aligning a central point of a left end face of the probe with an apex point of a dome-shaped cornea;
(c) depressing slowly the probe, along with a gradual increase of applanation force, on the display storage being displayed a half-loop or loop applanation image; and
(d) uniformizing a loop width of the half-loop or loop applanation image.
